(19) United States
(12) Patent Application Publication
(10) Pub. No.: US 2017/0168065 A1
(43) Pub. Date: Jun. 15, 2017

(54) METHODS AND COMPOSITIONS FOR IMAGING VIA MOLECULAR BIASING

(71) Applicant: LIGHT POLYMERS HOLDING, Grand Cayman, KY (US)

(72) Inventors: Evgeny Morozov, Burlingame, CA (US); Mary Parent, Mountain View, CA (US); Michael Joseph Katila, Redwood City, CA (US); Chris Micklitsch, San Francisco, CA (US); Joseph Marc McConnaughey, Temecula, CA (US)

(21) Appl. No.: 15/378,788

(22) Filed: Dec. 14, 2016

Related U.S. Application Data
(60) Provisional application No. 62/266,792, filed on Dec. 14, 2015.

(51) Int. Cl. G01N 33/68; G01N 21/23; G01N 1/30

(52) U.S. Cl. CPC G01N 33/6803; G01N 33/6887; G01N 1/30; G01N 21/23; G01N 2333/78; G01N 2800/7047

(57) ABSTRACT

The present disclosure relates to methods of using a lyotropic liquid crystal to detect protein structure; devices that can perform those methods described herein; and compositions that include a biospecimen and a composition that includes a birefringent small molecule or a birefringent polyaramide, where the birefringent small molecule and/or polyaramide coating solution exhibit a lyotropic liquid crystal phase.

### CONTINUING APPLICATION DATA

This application claims the benefit of U.S. Provisional Application Ser. No. 62/266,792, filed Dec. 14, 2015, which is incorporated by reference herein.

### BACKGROUND

Protein fibrils have been identified as a major cause of many vascular and neurological degenerative diseases by the global medical community. The only diagnostic tools available to the medical community today are neutron scattering, scanning electron microscopy, and dye-based injection imaging, which are expensive, limited in availability, and can be inaccurate. There are many drug options to treat these diseases but the health insurance industry requires more acceptable and lower cost diagnostics methods. There is a need for much simpler, less invasive detection at the earlier stages of these types of diseases that is affordable and acceptable to health care insurance companies.

### SUMMARY

The present disclosure relates to methods and compositions of creating or observing structural molecular biasing of a lyotropic liquid crystal by a multiprotein complex.

In one aspect, the methods include forming a layer that includes a biospecimen and forming a layer that includes a composition that includes a birefringent small molecule or a birefringent polyaramide. The layer including a biospecimen can include a protein concentrated from a biospecimen and/or a multiprotein complex. The multiprotein complex can include a fibril. The layers can be formed in any order, including simultaneously, that allows molecular biasing of a lyotropic liquid crystal by a multiprotein complex.

The methods can include imaging the layers. The imaging may be performed and/or analyzed by imaging software. Imaging the layers can include exposing the layers to polarized light and/or magnifying the layers. The methods can further include analyzing structural molecular biasing of a lyotropic liquid crystal by the biospecimen.

The methods can be performed by a device including, for example, a smartphone. The smartphone can include imaging software and/or analytical software. The device can include software that provides a health risk factor output.

In another aspect, a composition can include a biospecimen and/or a fibril-forming protein; and a composition that comprises a birefringent small molecule or a birefringent polyaramide.

These and various other features and advantages will be apparent from a reading of the following detailed description.

### BRIEF DESCRIPTION OF THE FIGURES

FIG. 1 shows an exemplary smartphone imaging adapter.

FIG. 2 shows an exemplary personal computer (PC)-linked, polarized, desktop microscope.

FIG. 3 shows a sample slide diagram.

FIG. 4 shows an exemplary schematic of a smartphone application interface.

FIG. 5 shows an exemplary health risk factor output. In some embodiments, software (e.g., an app) can analyze an image and determine a likelihood of risk related to an inquired disease.

FIG. 6 shows an exemplary schematic of a sampling, plating, and device interface.

FIG. 7A shows an exemplary image of collagen drops having the indicated concentrations plated on a slide without a poly(2,2′-disulfo-4,4′-benzidine terephthalamide) coating and visualized without the use of polarizers.

FIG. 7B shows an exemplary image of collagen drops having the indicated concentrations plated on a slide with a 4 wt % poly(2,2′-disulfo-4,4′-benzidine terephthalamide) coating and visualized without polarizers.

FIG. 7C shows an exemplary image of collagen drops having the indicated concentrations plated on a slide without a poly(2,2′-disulfo-4,4′-benzidine terephthalamide) coating, the image was obtained while the slide was placed between crossed polarizers.

FIG. 7D shows an exemplary image of collagen drops having the indicated concentrations on a slide with a 4 wt % poly(2,2′-disulfo-4,4′-benzidine terephthalamide) coating; the image was obtained while the slide was placed between crossed polarizers.

FIG. 8A shows an exemplary image of a 0.01 wt % collagen drop plated on a slide without a poly(2,2′-disulfo-4,4′-benzidine terephthalamide) coating, visualized using polarizers, 100× magnification.

FIG. 8B shows an exemplary image of a 0.01 wt % collagen drop plated on a slide with a poly(2,2′-disulfo-4,4′-benzidine terephthalamide) coating, visualized using polarizers, 100× magnification.

FIG. 8C an exemplary image of a 0.01 wt % collagen drop plated on a slide without a poly(2,2′-disulfo-4,4′-benzidine terephthalamide) coating; the image was obtained while the slide was placed between crossed polarizers, 100× magnification.

FIG. 8D is an exemplary image of a 0.01 wt % collagen drop plated on a slide with a poly(2,2′-disulfo-4,4′-benzidine terephthalamide) coating; the image was obtained while the slide was placed between crossed polarizers, 100× magnification.

FIG. 8E shows a slide with a poly(2,2′-disulfo-4,4′-benzidine terephthalamide) coating but no collagen; the image was obtained while the slide as placed between crossed polarizers, 100× magnification.

FIG. 8F shows an exemplary image of a 0.0006 wt % collagen drop plated on a slide with a poly(2,2′-disulfo-4,4′-benzidine terephthalamide) coating; the image was obtained while the slide was placed between crossed polarizers, 100× magnification.

FIG. 8G shows an exemplary image of a 0.0025 wt % collagen drop plated on a slide with a poly(2,2′-disulfo-4,4′-benzidine terephthalamide) coating; the image obtained while the slide as placed between crossed polarizers, 100× magnification.

FIG. 8H shows an exemplary image of a 0.01 wt % collagen drop plated on a slide with a poly(2,2′-disulfo-4,4′-benzidine terephthalamide) coating; the image was obtained while the slide as placed between crossed polarizers, 100× magnification.

FIG. 8I shows an exemplary image a slide with a poly(2,2′-disulfo-4,4′-benzidine terephthalamide) coating but no collagen; the image was obtained while the slide was placed between crossed polarizers, 400× magnification.

FIG. 8J shows an exemplary image of a 0.0006 wt % collagen drop plated on a slide with a poly(2,2′-disulfo-4,4′-benzidine terephthalamide) coating; the image was obtained while the slide was placed between crossed polarizers, 400× magnification.

FIG. 8K shows an exemplary image of a 0.0025 wt % collagen drop plated on a slide with a poly(2,2′-disulfo-4,4′-benzidine terephthalamide) coating; the image obtained while the slide as placed between crossed polarizers, 400× magnification.

FIG. 8L shows an exemplary image of a 0.01 wt % collagen drop plated on a slide with a poly(2,2′-disulfo-4,4′-benzidine terephthalamide) coating; the image was obtained while the slide as placed between crossed polarizers, 400× magnification.

### DETAILED DESCRIPTION

In the following detailed description, reference is made to the accompanying drawings that form a part hereof, and in which are shown by way of illustration several specific embodiments. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range.

As used in this specification and the appended claims, the singular forms “a,” “an,” and “the” encompass embodiments having plural referents, unless the content clearly dictates otherwise.

As used in this specification and the appended claims, the term “or” is generally employed in its sense including “and/or” unless the content clearly dictates otherwise.

As used herein, “have,” “having,” “include,” “including,” “comprise,” “comprising,” or the like are used in their open ended sense, and generally mean “including, but not limited to”. It will be understood that “consisting essentially of”, “consisting of”, and the like are subsumed in “comprising,” and the like.

In this disclosure:

“birefringent” refers to the optical property of a material having a refractive index that depends on the polarization and/or propagation direction of light be transmitted therethrough;

“refractive index” or “index of refraction” refers to the absolute refractive index of a material that is understood to be the ratio of the speed of electromagnetic radiation in free space to the speed of the radiation in that material. The refractive index can be measured using known methods and is generally measured using an Abbe refractometer in the visible light region (available commercially, for example, from Fisher Instruments of Pittsburgh, Pa.). It is generally appreciated that the measured index of refraction can vary to some extent depending on the instrument;

“substantially transparent” refers to a material that transmits at least 90%, at least 95%, or at least 98% of incident visible light excluding reflections at the interfaces (e.g., due to index mismatches) light transmittance values can be measured using ASTM methods and commercially available light transmittance instruments;

“biospecimen” refers to a material isolated from an animal including, for example, a human, and can include, for example, tissue, blood, plasma, urine, cerebrospinal fluid, tissue, etc., or, in some embodiments, a concentrate or isolate thereof. A biospecimen may be collected and/or isolated.

“fibril” refers to a small or fine fiber or filament. In some embodiments, a fibril can have a diameter of 1 to 200 nm. In some embodiments, the fibril is formed by the aggregation of a protein.

The present disclosure relates to methods of using a lyotropic liquid crystal to detect a multiprotein complex. The pattern, structure, and/or orientation of the lyotropic liquid crystal is influenced and/or altered by multiprotein complex formation which can be, in turn, influenced and/or altered by protein orientation and/or fibril formation. The methods can include forming a layer that includes a biospecimen and forming a layer that includes a birefringent small molecule or a birefringent polyaramide. The birefringent small molecule and/or polyaramide coating solution exhibit a lyotropic liquid crystal phase. In some embodiments, the layers are formed sequentially, one atop another. In some embodiments, the layers are formed simultaneously by, for example, mixing the compositions and forming a single layer.

The multiprotein complexes can include any protein which associates with another protein to form a multiprotein complex. The proteins which form the protein complex may be identical or different. In a preferred embodiment, a multiprotein complex is formed by identical proteins that associated into a fibrillar structure, at the nanometer scale. In some embodiments, a protein that associates into a multiprotein complex can include one or more of the proteins listed in Table 1 or Table 2. In some embodiments, it is preferred that the multiprotein complex forms fibrils.

TABLE 1

| DISEASE: | MOLECULE: | BODY LOCATION: |
|---|---|---|
| RHEUMATOID ARTHRITIS | Serum Amyloid | Blood/Plasma |
| | A | |
| ATHEROSCLEROSIS | Apolipoprotein | Blood/Plasma |
| (HEART DISEASE) | A1 | |
| FAMILIAL AMYLOIDAL | Transthyretin | Blood/Plasma |
| CARDIOMYOPATHY (FAC) | | |
| AMYLOIDOSIS | Light-chain | Blood/Plasma |
| | Anitbodies | |
| DIALYSIS-ASSOCIATED | β-2 | Blood/Plasma |
| AMYLOIDOSIS | Microglobulin | |
| TYPE II DIABETES | Islet Amyloid | Pancreas/Plasma |
| | Polypeptide | |

TABLE 2

| DISEASE: | MOLECULE: | BODY LOCATION: |
|---|---|---|
| ALZHEIMER'S DISEASE | A-β (1-42) | Cerebrospinal Fluid |
| | | (CSF) |
| CHRONIC TRAUMATIC | Lewy Bodies | CSF |
| ENCEPHALOPATHY (CTE) | (α-Synuclein) | |
| PARKINSON'S DISEASE | α-Synuclein | CSF |
| HUNTINGTON'S DISEASE | Huntingtin | CSF |
| FAMILIAL AMYLOIDAL | Transthyretin | CSF |
| POLYNEUROPATHY(FAP) | | |

In a preferred embodiment, an observable pattern, structure, and/or orientation of the lyotropic liquid crystal is influenced and/or altered by multiprotein complex formation and multiprotein complex formation is influenced and/or altered by protein orientation and/or fibril formation that can be correlated with a disease state or risk of a disease.

The present disclosure further relates to devices that can perform the methods described herein. In some embodiments, the devices may be portable and, for example, adapted for use with a smartphone. In some embodiments the devices include imaging and/or analytical software for acquiring and analyzing images of the layers formed using the methods described herein. In some embodiments the devices include software that recognizes structural patterns that are presented in the images.

The present disclosure also relates to compositions that include a biospecimen and a composition that includes a birefringent small molecule or a birefringent polyaramide, where the birefringent small molecule and/or polyaramide coating solution exhibit a lyotropic liquid crystal phase. The biospecimen includes a purified biospecimen and/or one or more proteins isolated or concentrated from a biospecimen.

The methods described herein permit detection of structural molecular biasing of birefringent lyotropic liquid crystals induced by protein fibrils or other protein structures and orientations. A multiprotein complex (even at a nanometer scale), when in contact with birefringent lyotropic liquid crystals, can drive molecular biasing of the birefringent lyotropic liquid crystals. Without wishing to be bound by theory, it is believed that the structural orientation or molecular biasing spreads to a micrometer scale area due to correlation of the intermolecular interactions in the liquid crystal, making the structural molecular biasing observable in polarized light and permitting visualization of the structure without the need for sophisticated high power microscopy and imaging techniques.

As described further in Example 1, a liquid crystalline polyaramide (poly(2,2′-disulfo-4,4′-benzidine terephthalamide)) can be aligned to reflect the structure and/or orientation of a coated, dried layer of assembled protein fibrils. After deposition of a liquid crystal phase on an existing biomolecular layer by any method, application of shear stress to the coating liquid preorganizes the liquid crystal that then allows for protein fibril-based molecular reorganization of the polyaramide. Drying of the coating liquid results in crystallization of the polyaramide material possessing molecular ordering biased by the fibrils' assembled structure. The biased, crystallized polyaramide structure is easily observed using only 100× magnification in polarized light.

In some embodiments, a biospecimen and/or a protein concentrated from a biospecimen may be used to form a layer prior to the formation of a layer that includes a composition that includes a birefringent lyotropic liquid crystal. In some embodiments, a biospecimen may be mixed with a composition that includes birefringent lyotropic liquid crystal prior to forming a layer.

In some embodiments, the layer may be formed on a substrate. In some embodiments, the substrate may be optically clear and/or substantially transparent. In some embodiments, the substrate is glass. In some embodiments, at least one of the formed layers has a surface that is in direct contact with the substrate.

In some embodiments, a protein may be isolated or concentrated from a biospecimen and used to form a layer prior to the addition of a composition that includes a birefringent lyotropic liquid crystal material. In some embodiments, a protein isolated or concentrated from a biospecimen may be mixed with a composition that includes birefringent lyotropic liquid crystal prior to forming a layer.

In some embodiments the concentration of the protein in the formed layer may be less than about 0.1 percent (%), less than about 0.05%, less than about 0.01%, less than about 0.005%, or less than about 0.001%. In some embodiments the concentration of the protein in the formed layer may be up to about 0.1%, up to about 1%, up to about 2%, up to about 5%, or up to about 10%. In some embodiments, the protein is a fibril-forming protein. In some embodiments, the formed layer is substantially free of lipids.

A protein may be isolated or concentrated from a biospecimen by any suitable means including, for example, by electrophoresis, chromatography and/or immunoprecipitation.

In some embodiments, a composition that includes a birefringent lyotropic liquid crystal material is a solution. The birefringent small molecule and/or polyaramide coating solution exhibits a lyotropic liquid crystal phase. In some embodiments, a coating solution can be at least 75% wt, at least 80% wt, at least 85% wt, or at least 90% wt water. In some embodiments, a coating solution can be at least 1% wt, at least 5% wt, at least 10% wt, at least 15% wt lyotropic liquid crystal. In some embodiments, a coating solution can be up to 10% wt, up to 15% wt, up to 20% wt, or up to 25% wt lyotropic liquid crystal material. In many embodiments the coating solution is from 1 to 25% wt, from 1 to 20% wt, from 1 to 15% wt, or from 1 to 10% wt lyotropic liquid crystal material. Shear coating allows the coating solution to be aligned according to the coating direction.

In some embodiments, the biospecimen and composition that comprises a birefringent small molecule or a birefringent polyaramide form a composition. In some embodiments, a protein and/or composition that comprises a birefringent small molecule or a birefringent polyaramide form a composition. The composition may have one or more layers. In some embodiments, the protein is concentrated or isolated from a biospecimen. In some embodiments, the protein is a fibril-forming protein. In a preferred embodiment, the protein is part of a multiprotein complex.

This disclosure further relates to analyzing a layer and/or layers of a biospecimen and a composition that comprises a birefringent small molecule or a birefringent polyaramide to determine the presence and/or amount of structural molecular biasing of a lyotropic liquid crystal by the biospecimen. In some embodiments, the structural molecular biasing of the lyotropic liquid crystal may be analyzed using a device and/or software, as further described below. In some embodiments, the presence and/or amount of structural molecular biasing is related to and can be correlated with the concentration of a fibril-forming protein and/or a multiprotein complex.

Birefringence described herein refers to macroscopic birefringence or molecular level birefringence. For example, coating the polyaramides or small molecules (e.g., as described herein) by any type of shear coating can align the molecules in more or less the same direction over a macroscopic dimension and exhibit a macroscopic birefringence. Birefringence can be characterized by measuring a refractive index of the three principal refractive indices (nₓ, n_{y} and n_{z}) associated with the Cartesian coordinate system related to the deposited birefringent polyaramide or small molecule layer or the corresponding major surface of the retarder film or plate. Two principal directions for refractive indices nₓ and n_{y} may belong to the xy-plane coinciding with a plane of the retarder, while one principal direction for refractive index (n_{z}) coincides with a normal line to the retarder.

### Birefringent Lyotropic Liquid Crystals

The birefringent polymers can be made from various base materials having suitable optical birefringent and other properties, such as thermal resistance, light transmittance, and the like. In some embodiments, the birefringent polymers are water-soluble and exhibit a liquid crystal phase in water. In some embodiments, the birefringent polymers can be dissolved in an organic solvent including, for example, chloroform, toluene, acetonitrile, etc. The birefringent polymers can be deposited, or coated onto a substrate via a solution including, for example, an aqueous solution. Once coated or deposited the aligned birefringent polymers can be stabilized or made less solvent-soluble by cross-linking or by ion exchange, generally termed “passivation.”

An exemplary birefringent lyotropic liquid crystal polymer is a birefringent polyaramide that exhibits a lyotropic liquid crystal phase having the following formula:

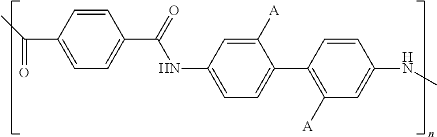

wherein:
A is independently selected from SO₃H or COOH, or their salt of an alkali metal, ammonium, quaternary ammonium, alkali earth metal, Al³⁺, La³⁺, Fe³⁺, Cr³⁺, Mn²⁺, Cu²⁺, Zn²⁺, Pb²⁺ or Sn²⁺; and n is an integer from 2 to 10,000. In one embodiment, the number-average molecular weight is about 10,000 to about 150,000. In another embodiment, the number-average molecular weight is about 50,000 to about 150,000.

In many embodiments the birefringent polyaramide is a polymer of a formula below:

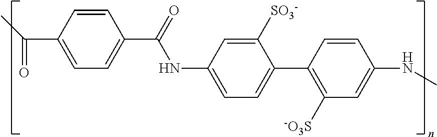

wherein n is an integer in a range from 2 to 10,000 or from 5 to 2000. In one embodiment, the number-average molecular weight is about 10,000 to about 150,000. In another embodiment, the number-average molecular weight is about 50,000 to about 150,000. This polymer is referred to as: poly(2,2′-disulfo-4,4′-benzidine terephthalamide) and can be a sodium or ammonium salt thereof. An example of a synthesis of this polymer is described in U.S. Pat. No. 8,512,824. A birefringent polyaramide film or layer formed from this polymer is birefringent and has the following refractive indices: nₓ=1.84, n_{y}=n_{z}=1.58 (at 550 nm), where nₓ and n_{y} correspond to two mutually perpendicular directions in a plane and n_{z} corresponds to the normal direction to the plane.

An exemplary birefringent lyotropic liquid crystal polymer is a birefringent polymer that can exhibit a lyotropic liquid crystal phase having the following formula:

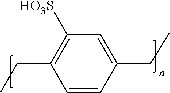

or a salt thereof, wherein n is an integer in a range from 25 to 10,000. This polymer is referred to as poly(monosulfo-p-xylene) or a salt thereof. The compound has the following refractive indices: nₓ=1.71, n_{y}=n_{z}=1.53 (at 550 nm).

This polymer can be synthesized as follows:

300 ml of sulfuric acid was added to 212 g of p-xylene at 90° C. The reaction mass was stirred at 90-100° C. for 30 min then cooled to 20-25° C. and poured into a beaker with 500 g of mixture of water and ice. The resulting suspension was separated by filtration and the filter cake rinsed with cool (5° C.) solution of 300 ml of hydrochloric acid in 150 ml of water.

The material was vacuum dried at 50 mbar and 50° C. for 24 hrs. Yield of 2,5-dimethylbenzenesulfonic acid was 383 g (contained 15% water).

92.6 g of 2,5-dimethylbenzenesulfonic acid was added to 1700 ml of chloroform and the mixture was purged with argon gas. Then it was heated to boiling with a 500 W lamp placed right against the reaction flask so that stirred contents of the flask was well lit. 41 ml bromine in 210 ml of chloroform was added dropwise within 4-5 hrs to the agitated boiling mixture. Once all bromine had been added the light exposure with refluxing continued for an extra hour. 900 ml of chloroform was distilled and the reaction mass was allowed to cool overnight. Precipitated material was isolated by filtration, the filter cake was rinsed with 100 ml of chloroform, squeezed and recrystallized from 80 ml of acetonitrile. Yield of 2,5-bis(bromomethyl)benzenesulfonic acid was 21 g.

4.0 g of sodium borohydride in 20 ml of water was added to a stirred mixture of 340 mg of CuCl₂, 10.0 g of 2,5-bis(bromomethyl)benzenesulfonic acid, 10.4 g of sodium bromide, 45 ml of amyl alcohol and 160 ml of degassed water and the reaction mass was agitated for 10 min. Then the mixture was transferred to a 1-liter separatory funnel, 300 ml of water was added and after shaking the mixture was allowed to stand for an hour. The bottom layer was isolated, clarified by filtration and ultrafiltered using a polysulfone membrane with 10,000 molecular weight cut-off. Yield of polymer (Na salt) is 4.0 g (on dry basis). An aqueous solution of this material was coated onto a glass substrate with a Mayer rod and dried.

### Birefringent Small Molecules

The birefringent small molecules can be made from various base materials having suitable optical birefringent and other properties, such as thermal resistance, light transmittance, and the like. The birefringent small molecules are water-soluble and exhibit a liquid crystal phase in water. The birefringent small molecules can be deposited, or coated onto a substrate via an aqueous solution. Once coated or deposited the aligned birefringent small molecules can be stabilized or made less water-soluble by ion exchange, generally termed “passivation.”

An exemplary birefringent lyotropic liquid crystal is a birefringent small molecule that exhibits a lyotropic liquid crystal phase having the following formula:

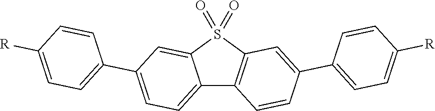

wherein:
R is independently selected from SO₃H or COOH, or their salt of an alkali metal, ammonium, quaternary ammonium, alkali earth metal, Al³⁺, La³⁺, Fe³⁺, Cr³⁺, Mn²⁺, Cu²⁺, Zn²⁺, Pb²⁺ or Sn²⁺.

In many embodiments the birefringent small molecule has the formula below:

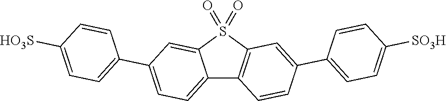 [SMILES: O=S(=O)(O)c1ccc(-c2ccc3c(c2)S(=O)(=O)c2cc(-c4ccc(S(=O)(=O)O)cc4)ccc2-3)cc1]

This is referred to as 4,4′-(5,5′-dioxidodibenzo[b,d]thiene-3,7-diyl)dibenzenesulfonic acid). Examples of synthesis of this small molecule are described in U.S. Publication No. 2012/0113380. A birefringent film or layer formed from this small molecule is birefringent and has the following refractive indices: nₓ=1.51, n_{y}=1.87, n_{z}=1.73 (at 550 nm), where nₓ and n_{y} correspond to two mutually perpendicular directions in a plane and n_{z} corresponds to the normal direction to the plane.

Another exemplary birefringent lyotropic liquid crystal is a birefringent small molecule having the following formula:

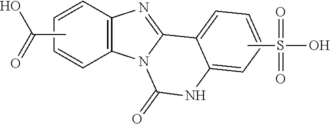

or a salt thereof. This small molecule is referred to as 2(3)-sulfo-6,7-dihydrobenzimidazo[1,2-c]quinazoline-6-one-9(10)-carboxylic acid. Examples of synthesis of this small molecule are described in U.S. Publication No. 2010/0039705. A birefringent film or layer formed from this small molecule is birefringent and has the following refractive indices: nₓ=1.51, n_{y}=1.87, n_{z}=1.72 (at 550 nm), where nₓ and n_{y} correspond to two mutually perpendicular directions in a plane and n_{z} corresponds to the normal direction to the plane.

A further exemplary birefringent lyotropic liquid crystal is a birefringent small molecule having the following formula:

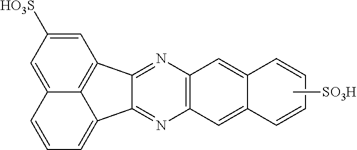

or a salt thereof. This small molecule is referred to as acenaphtho[1,2-b]benzo[9]quinoxaline bisulfonic acid. A birefringent film or layer formed from this small molecule is birefringent and has the following refractive indices: nₓ=1.56, n_{y}=1.89, n_{z}=1.77 (at 550 nm), where nₓ and n_{y} correspond to two mutually perpendicular directions in a plane and n_{z} corresponds to the normal direction to the plane.

This birefringent small molecule can be synthesized as follows:

5.82 g of acenaphthoquinone (27.44 mmol) and 5.0 g of naphthalene-2,3-diamine (31.6 mmol) were added to 200 ml of acetic acid and the resulting suspension was stirred at room temperature for 6 hrs. Then the reaction mixture was filtered through fiberglass filter (D=80 mm) and filter cake was washed with 100 ml of acetic acid, then with 1000 ml of water and dried at 100-105° C. for 24 hrs. Yield of acenaphtho[1,2-b]benzo[9]quinoxaline was 8.7 g.

8.5 g of acenaphtho[1,2-b]benzo[9]quinoxaline was added to 60 ml of 30% oleum with agitation at <50° C. The reaction was heated to 75° C., agitated at temperature for 2 hours and then allowed to cool to room temperature.

132 ml of water was added with agitation at <50° C. and the resulting suspension agitated overnight.

Precipitated matter was isolated by filtration, washed with 1 L of glacial acetic then with 500 ml of acetone and air dried at 100-110° C. for 7 hrs. Yield of acenaphtho[1,2-b]benzo[9]quinoxaline bisulfonic acid was 13.2 g.

In many embodiments two or more of the birefringent small molecules described above can be combined to form a mixture of birefringent small molecules. For example, 2(3)-sulfo-6,7-dihydrobenzimidazo[1,2-c]quinazoline-6-one-9(10)-carboxylic acid or a salt thereof can be combined with 4,4′-(5,5′-dioxidodibenzo[b,d]thiene-3,7-diyl)dibenzenesulfonic acid) or a salt thereof. In some embodiments, mixtures of the small molecules may improve the structural molecular biasing driven by a multiprotein complex.

### Devices

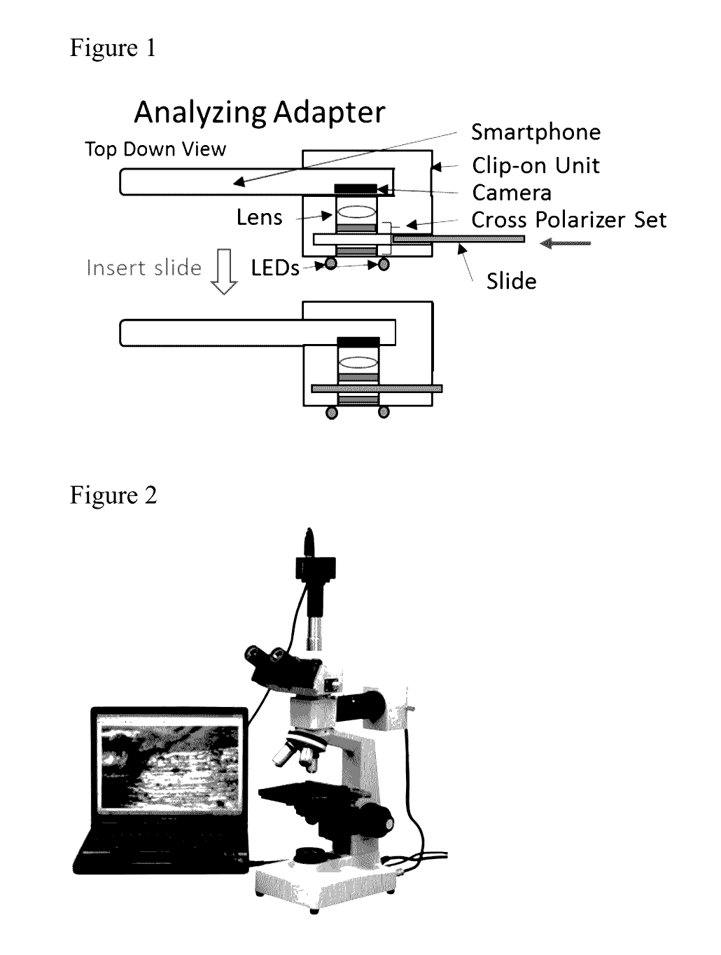

In some embodiments, a method of using a lyotropic liquid crystal to detect protein structure, may be performed by a device. In some embodiments, the device can include a camera including, for example, a smartphone camera, a set of cross-polarizers, and a magnification lens. In some embodiments, the camera, magnification lens, and cross-polarizers can be part of a smartphone camera adapter, as shown in one embodiment in FIG. 1. In some embodiments, the magnification lens provides 80-400× magnification or 80-100× magnification. In some embodiments, the device further includes an adjustable LED light-source.

In some embodiments, the device can include a digital PC-linked, polarized, desktop microscope that includes a set of cross-polarizers, as shown in one embodiment in FIG. 2.

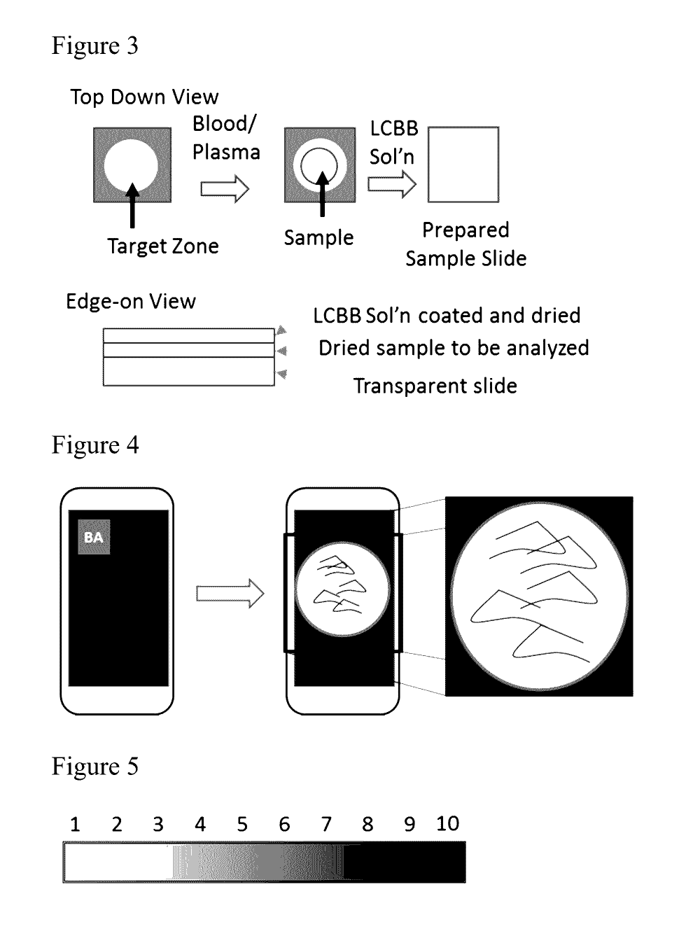

In some embodiments, the device can include a transparent sample slide on which the sample and a lyotropic liquid crystal solution are coated. In a preferred embodiment, the sample slide is optically transparent. The device can further include a sample slot for the slide and the slide can be configured to be placed in sample slot. The device can, in some aspects, include a reservoir of a composition that includes a birefringent lyotropic liquid crystal, including for example, a lyotropic liquid crystal solution. In some embodiments, the lyotropic liquid crystal solution may be referred to as a liquid crystal biomolecular biasing solution (LCBB). Such a slide and a method of preparing the sample are shown in one embodiment in FIG. 3.

In some embodiments, the device can include a receptacle for a biospecimen. In some embodiments, the biospecimen can be loaded into a cartridge. The biospecimen can include, for example, blood, a blood filtrate, plasma, cerebrospinal fluid, a tissue sample, etc. In some embodiments, the cartridge can process the biospecimen, including, for example, a sample of blood and/or plasma, to be suitably devoid of blood cells and other potential interfering bodies. In some embodiments, the coating of the biospecimen and/or a composition that comprises a birefringent small molecule or a birefringent polyaramide onto a slide can occur in the cartridge.

In some embodiments, the device can include a shear-coating device.

In some embodiments, the device can include an imaging and/or analysis application and/or software. The software can be smartphone software. The application can, for example, analyze the image obtained by, for example, comparing the image against control images, calculating vectors, etc. The application can, in some embodiments, enhance the image. The application can, in some embodiments, include an image structure analysis program. The application can include a user interface as shown in one embodiment in FIG. 4. The application can, in some embodiments, provide a health risk factor output, as shown in one embodiment, in FIG. 5. In some embodiments, the application can provide a health risk factor output for a disease listed in Table 1.

In some embodiments, a slide that has been processed in a cartridge, as described above, can be imaged, including for example, in a smartphone adapter device.

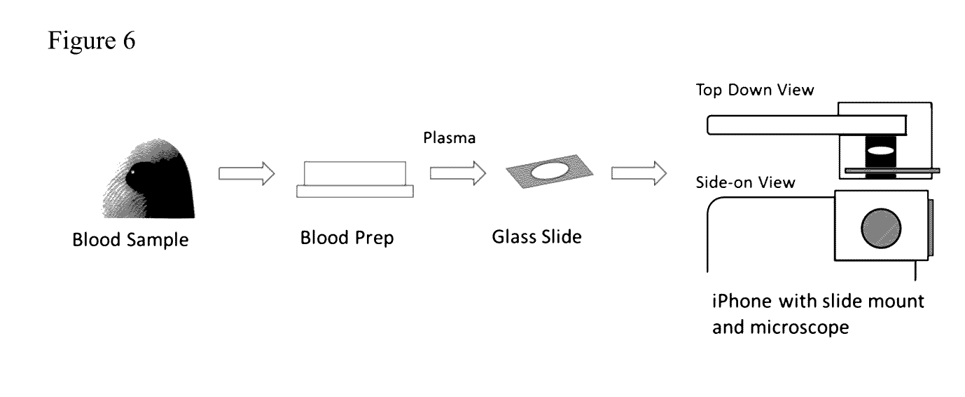

In some embodiments, a biospecimen can be loaded into the biomolecular imaging device. The biospecimen or an isolate or concentrate of the biospecimen can be placed and/or dried on the sample slide. In preferred embodiments, the device includes a shear coating mechanism. In some embodiments, the biospecimen and/or a composition including a lyotropic liquid crystal can be shear-coated. The biospecimen and the composition including a lyotropic liquid crystal may be coated sequentially or simultaneously. In some embodiments, a composition including a lyotropic liquid crystal can be shear-coated after the biospecimen has been placed and dried on the sample slide. In some embodiments, the prepared sample slide can be placed in the biomolecular imaging device such that it is positioned optimally in front of a camera and can thus be imaged, as shown in one embodiment in FIG. 6. In some embodiments, the image obtained from the slide can be analyzed by a software application which analyzes or assists in analyzing the image. The software application can, in some embodiments, be a smartphone software application. The software application can, in some embodiments, be a PC software program. In some embodiments, health information can be provided as an output, and/or the data can be securely stored on a cloud server. In some embodiments the health information provided relates to one of the disease listed in Table 1 or Table 2.

Objects and advantages of this disclosure are further illustrated by the following examples, but the particular materials and amounts thereof recited in these examples, as well as other conditions and details, should not be construed to unduly limit this disclosure.

### EXAMPLE

### Example 1—Detecting Structural Molecular Biasing of Collagen

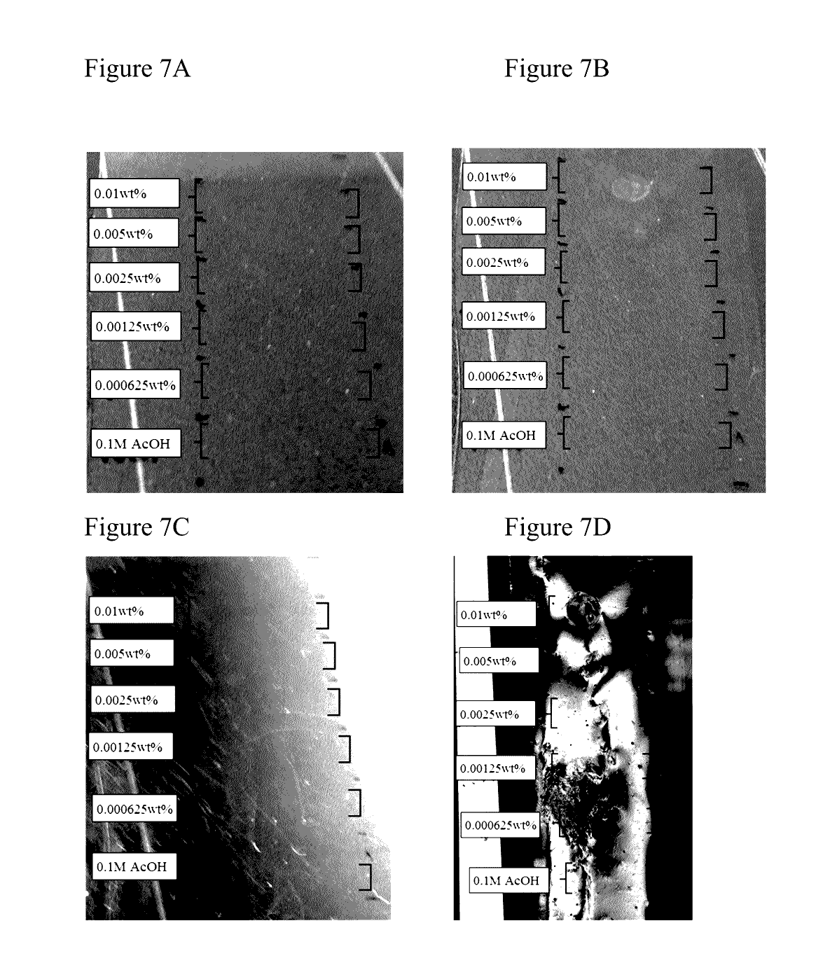

10 uL drops of 0.01 wt %, 0.005 wt %, 0.025 wt %, 0.00125 wt %, 0.000625 wt % of Collagen in 0.1M Acetic acid (AcOH) or 0.1M AcOH (control) were placed on a glass slide, then dried in 37° C. oven. After drying, the slide was rinsed with DI water and blow dried. The slide was then coated with 4 wt % poly(2,2′-disulfo-4,4′-benzidine terephthalamide), sodium form, using a 30 μm gap applicator, then dried in 37° C. oven. Results are shown in FIG. 7A-D.

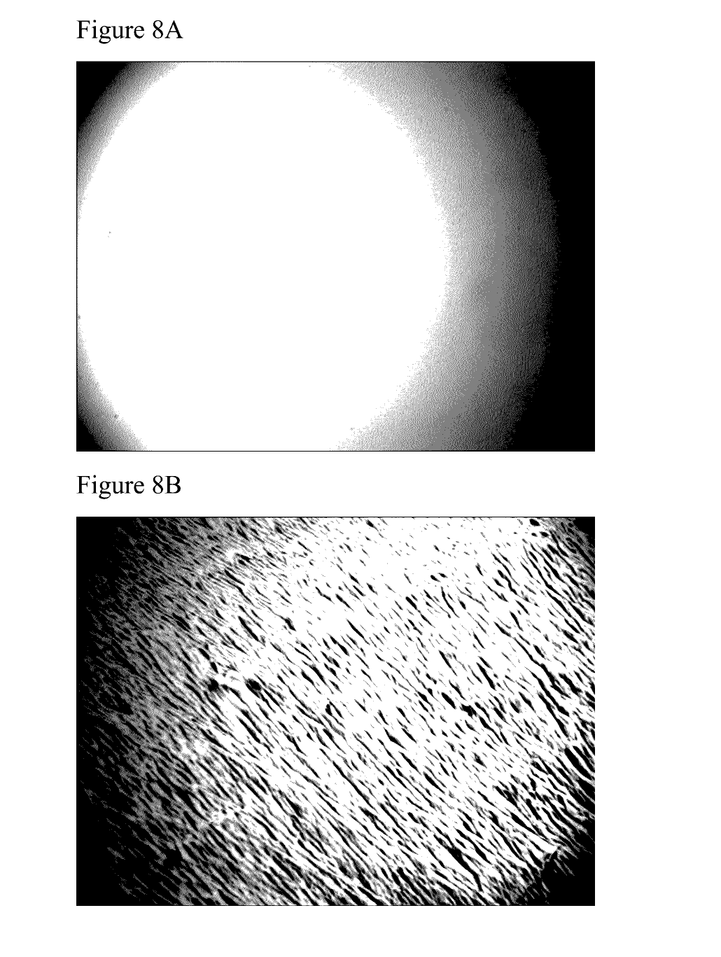

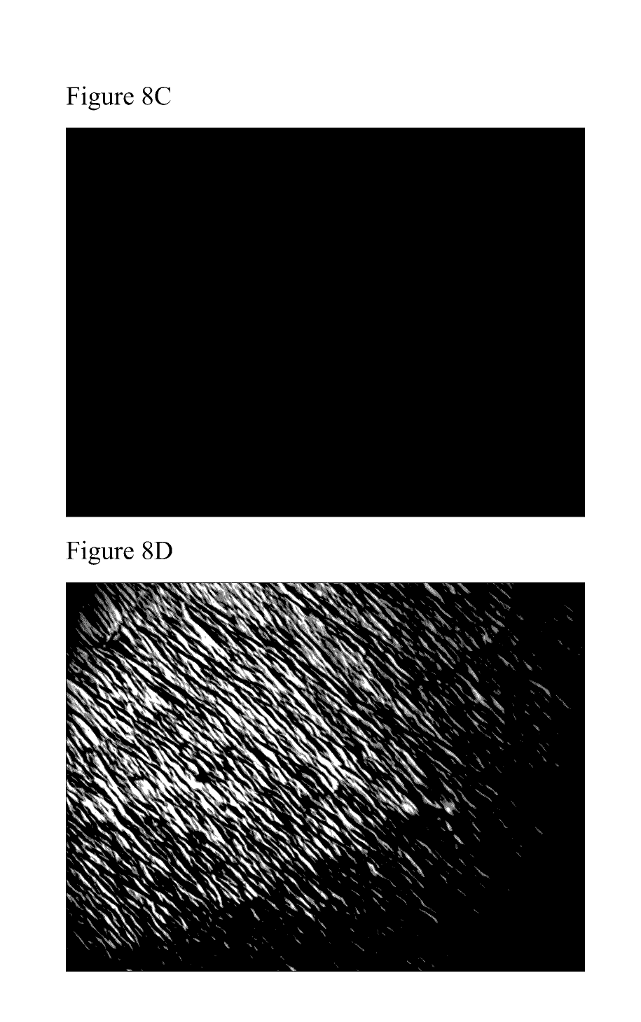

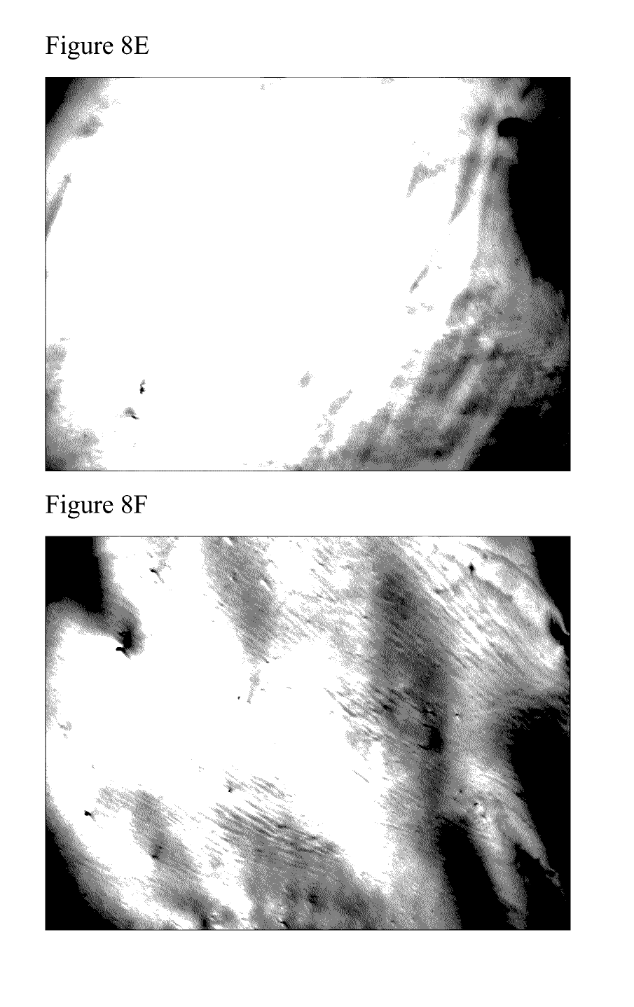

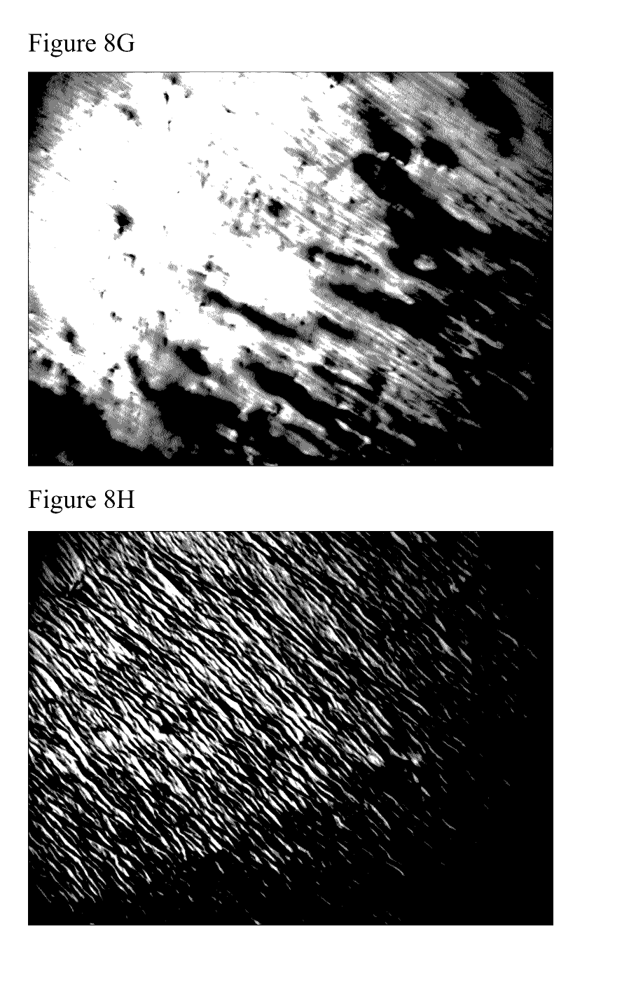

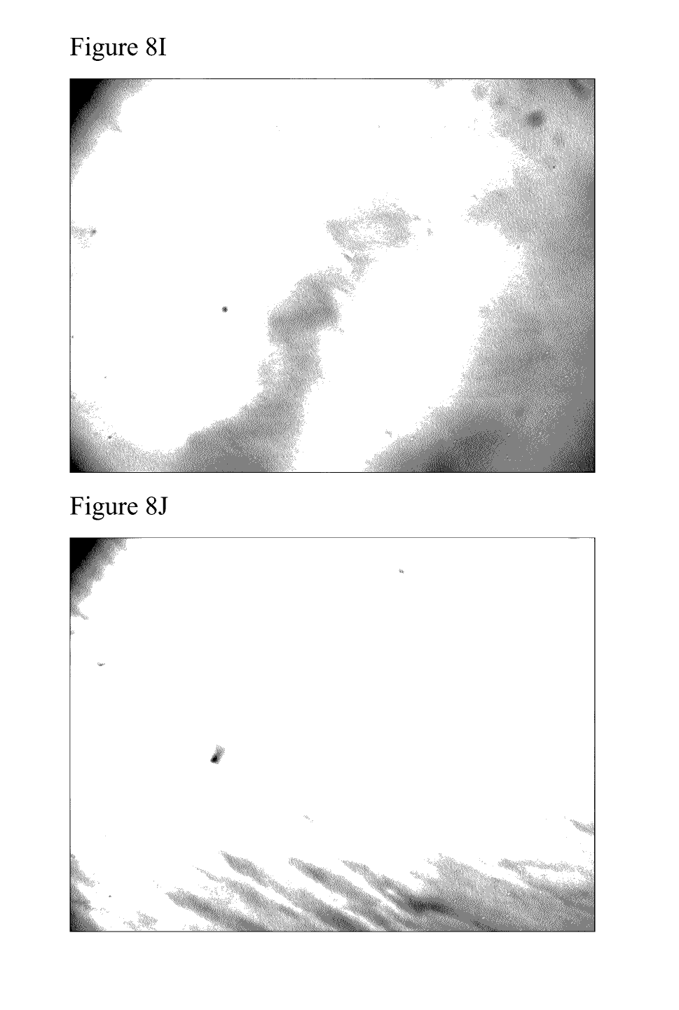

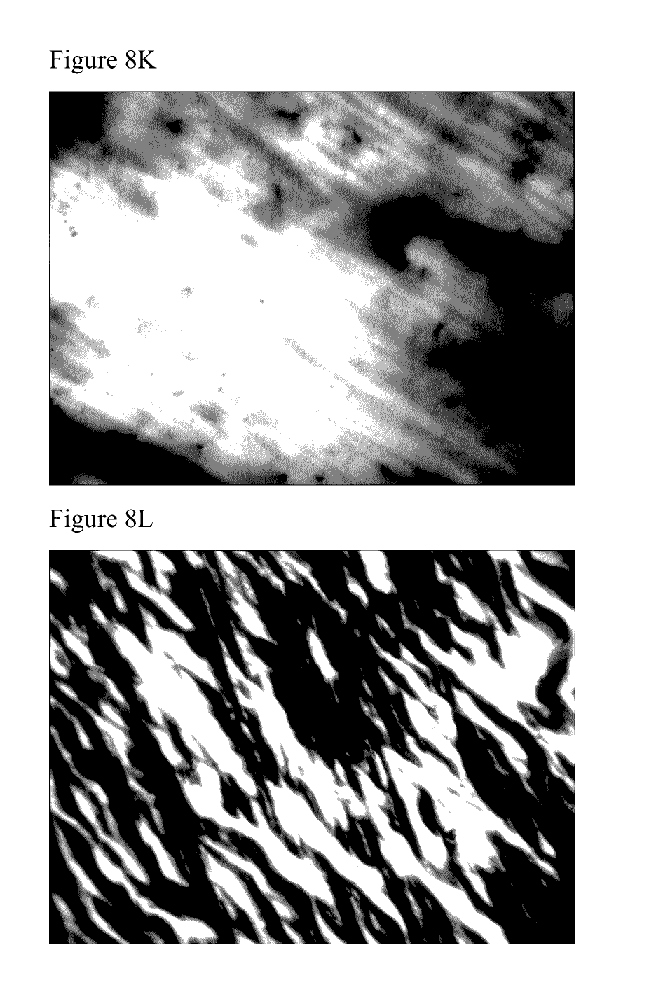

Without a poly(2,2′-disulfo-4,4′-benzidine terephthalamide) coating, the collagen drops were not visually obvious; however with a poly(2,2′-disulfo-4,4′-benzidine terephthalamide) coating, the drop areas appeared hazy, especially at higher collagen concentrations. Under high magnification, protein alignment is visible, as shown in FIGS. 8A-L.

Thus, embodiments of DETECTING STRUCTURAL MOLECULAR BIASING are disclosed.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof. The disclosed embodiments are presented for purposes of illustration and not limitation.

What is claimed is:

1. A method comprising
forming a layer comprising a biospecimen; and
forming a layer comprising a composition that comprises a birefringent small molecule or a birefringent polyaramide.

2. The method of claim 1, wherein the layer comprising a biospecimen comprises a protein concentrated from a biospecimen.

3. The method of claim 1, wherein the layer comprising a biospecimen is dried to form a first layer prior to forming the layer comprising a composition that comprises a birefringent small molecule or a birefringent polyaramide.

4. The method of claim 3, wherein the layer comprising a composition that comprises a birefringent small molecule or a birefringent polyaramide is formed on a surface of the first layer.

5. The method of claim 1, wherein both layers are formed simultaneously and a single layer comprises the biospecimen and the composition that comprises a birefringent small molecule or a birefringent polyaramide.

6. The method of claim 1, wherein the surface of at least one of the layers contacts a substantially transparent substrate.

7. The method of claim 1, the method comprising shear coating the layer comprising a composition that comprises a birefringent small molecule or a birefringent polyaramide.

8. A method according to claim 1, wherein the birefringent small molecule comprises a molecule having at least one of the following formulas: 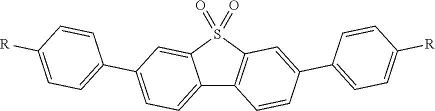
wherein:
R is independently selected from SO₃H or COOH, or their salt of an alkali metal, ammonium, quaternary ammonium, alkali earth metal, Al³⁺, La³⁺, Fe³⁺, Cr³⁺, Mn²⁺, Cu²⁺, Zn²⁺, Pb²⁺ or Sn²⁺, 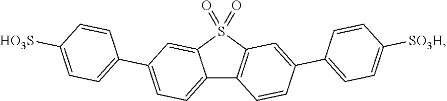 [SMILES: O=S(=O)(O)c1ccc(-c2ccc3c(c2)S(=O)(=O)c2cc(-c4ccc(S(=O)(=O)O)cc4)ccc2-3)cc1]
or a salt thereof; or 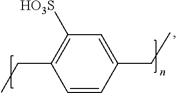
or a salt thereof
wherein n is an integer in a range from 25 to 10,000.

9. The method according to claim 1, wherein the birefringent polyaramide comprises a molecule having at least one of the following formulas: 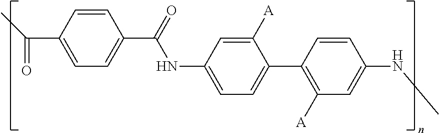
wherein:
A is independently selected from SO₃H or COOH, or their salt of an alkali metal, ammonium, quaternary ammonium, alkali earth metal, Al³⁺, La³⁺, Fe³⁺, Cr³⁺, Mn²⁺, Cu²⁺, Zn²⁺, Pb²⁺ or Sn²⁺, and
n is an integer from 2 to 10,000; or 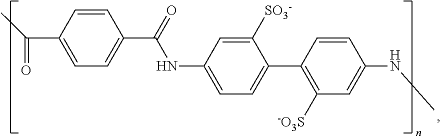
or a salt thereof.

10. The method of claim 1 further comprising imaging the layers.

11. The method of claim 10, wherein imaging the layers comprises exposing the layers to polarized light.

12. The method of claim 10, where imaging the layers comprises magnifying the layers.

13. The method of claim 1, the method further comprising analyzing structural molecular biasing of a lyotropic liquid crystal by the biospecimen.

14. A device that performs the method of claim 1.

15. The device of claim 14, wherein the device comprises a smartphone.

16. The device of claim 14, wherein the device comprises imaging software.

17. A composition comprising
a biospecimen; and
a composition that comprises a birefringent small molecule or a birefringent polyaramide.

18. A composition comprising
a fibril-forming protein; and
a composition that comprises a birefringent small molecule or a birefringent polyaramide.

19. A composition according to claim 17, wherein the birefringent small molecule comprises a molecule having at least one of the following formulas: 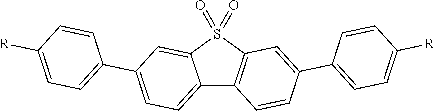
wherein:
R is independently selected from SO₃H or COOH, or their salt of an alkali metal, ammonium, quaternary ammonium, alkali earth metal, Al³⁺, La³⁺, Fe³⁺, Cr³⁺, Mn²⁺, Cu²⁺, Zn²⁺, Pb²⁺ or Sn²⁺; 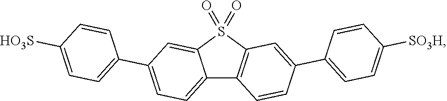 [SMILES: O=S(=O)(O)c1ccc(-c2ccc3c(c2)S(=O)(=O)c2cc(-c4ccc(S(=O)(=O)O)cc4)ccc2-3)cc1]
or a salt thereof; or 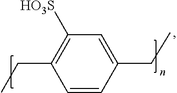
or a salt thereof
wherein n is an integer in a range from 25 to 10,000.

20. A composition according to claim 17, wherein the birefringent polyaramide comprises a molecule having at least one of the following formulas: 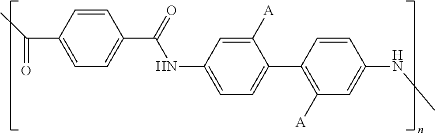
wherein:
A is independently selected from SO₃H or COOH, or their salt of an alkali metal, ammonium, quaternary ammonium, alkali earth metal, Al³⁺, La³⁺, Fe³⁺, Cr³⁺, Mn²⁺, Cu²⁺, Zn²⁺, Pb²⁺ or Sn²⁺, and
n is an integer from 2 to 10,000; or 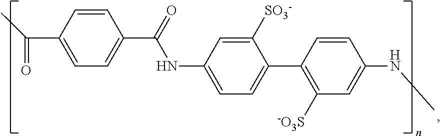
or a salt thereof.
